(19) United States
(12) Patent Application Publication
(10) Pub. No.: US 2022/0251638 A1
(43) Pub. Date: Aug. 11, 2022

(54) Methods to Identify Components in Nucleic Acid Sequences

(71) Applicant: Universal Sequencing Technology Corporation, Canton, MA (US)

(72) Inventors: Peiming Zhang, Gilbert, AZ (US); Ming Lei, Sharon, MA (US)

(21) Appl. No.: 17/595,758

(22) PCT Filed: May 27, 2020
(86) PCT No.: PCT/US2020/034759

§ 371 Date: Nov. 23, 2021

Related U.S. Application Data
(60) Provisional application No. 62/861,675, filed on Jun. 14, 2019.
(60) Provisional application No. 62/853,119, filed on May 27, 2019.

(51) Int. Cl. C12Q 1/6869

(52) U.S. Cl. CPC C12Q 1/6869

(57) ABSTRACT

This invention provides methods to identify or sequence a DNA or RNA molecule electronically in a single molecule level based on polymerase synthesis.

### CROSS-REFERENCE TO RELATED APPLICATIONS

This application claims priority to U.S. Provisional Application Ser. No. 62/853,119 filed May 27, 2019, and U.S. Provisional Application Ser. No. 62/861,675 filed Jun. 14, 2019, the entire disclosures of which are hereby incorporated herein by reference.

### FIELD

Embodiments of the present invention are related to methods and biochemical materials for an electronic sequencing device to read out individual nucleotides in a nucleic acid sequence using enzymes.

### THE PRIOR ART AND BACKGROUND

Throughout this application, various publications, patents, and patent applications to which this invention pertains have been referenced. The disclosures of these publications in their entireties are delineated as the state of the art. Also, in this application, the invention is illustrated primarily with DNA and DNA polymerase synthesis. The same mechanisms, principles, and features apply to RNA and RNA polymerase synthesis too with only slight modifications, e.g., replacing deoxyribonucleoside triphosphate (dNTP) with nucleoside triphosphate (NTP), replacing deoxyribose with ribose, and so on.

A prior art (U.S. Pat. Nos. 9,862,998 and 10,233,493) discloses a method to detect the nucleotide incorporation into DNA by monitoring the conformational changes of a DNA polymerase labeled with fluorescent dyes. Depending on the enzymes and dyes, different nucleotides, including those naturally occurring and modified, produced different amplitudes and durations for the fluorescent emissions. The method determines a nucleotide sequence from an ensemble of nucleic acid molecules.

Another prior art has demonstrated that a carbon nanotube charge sensor (FIG. 1) could electronically monitor the process of a single DNA polymerase incorporating individual naturally-occurring nucleotides into a DNA primer in real time¹ so that such an electronic device could potentially be used in the sequencing of nucleic acid polymers. However, the electric signals (appearing as spikes) cannot be effectively applied to identify individually incorporated nucleotides. As shown in the table below (adopted from Reference 1), there are overlaps between the characteristic parameters derived from the electric signals of the DNA polymerase incorporation overlap correspondingly among the naturally occurring nucleotides. Thus, neither τₗₒ, τₕᵢ, nor H values are sufficient to identify the incorporation of a particular dNTP with any degree of reliability.

| Template | Substrate | τₗₒ (ms) | τₕᵢ (ms) | H (nA) |
|---|---|---|---|---|
| poly(dT)₄₂ | dATP | 0.33 ± 0.08 | 71.4 ± 1.4 | 6.94 |
| poly(dA)₄₂ | dTTP | 0.42 ± 0.09 | 63.7 ± 1.1 | 4.90 |
| poly(dG)₄₂ | dCTP | 0.32 ± 0.07 | 39.0 ± 5.6 | 2.53 |
| poly(dC)₄₂ | dGTP | 0.33 ± 0.05 | 38.0 ± 5.8 | 2.40 |

τₗₒ: the duration of the time spent in the enzyme's closed conformation; τₕᵢ: the duration of the time spent in the enzyme’s open conformation; H: the average amplitude of each electric signal.

A prior art application (WO 2016/183218) claims that a mixture in which one or more of the native nucleotide triphosphates is replaced with an analog having a non-natural moiety that alters signal polarity in a distinguishable way without hurting the ability of the analog to base pair with its cognate nucleotide in a template strand during sequencing. As an example, α-thio-dATP results in a negative change in signal polarity and 2-thio-dTTP results in a positive change in signal polarity so that they can be used to distinguish between the T and A in a template by means of the said device charge sensor (see FIG. 2 for their structures). However, Weiss and coworkers have reported that the 2-thiopyrimidine-5′-triphosphate (2-thio-dNTP) analogs produced mixed behaviors in which the DNA polymerase I Klenow fragment (KF) activity produced negative excursions during 1 minute, and positive excursions during another minute.² That indicates that the modification on the Watson-Crick base pairing edge of a nucleobase causes uncertainty of the electronic signals, in turn resulting in uncertainties as to the determination of the nucleotide incorporation for the nucleic acid sequencing.

The enzymatic incorporation of nucleoside triphosphates to a DNA strand has a similar kinetic pathway between the mismatched and matched dNTPs³ as well as between different DNA polymerases⁴, as shown in FIG. 4. In general, the DNA polymerization catalyzed by DNA polymerases is a kinetically controlled process. There are several major steps involved in the process: (1) the conformation closing, (2) the triphosphate coupling to 3′ end of DNA, and (3) DNA translocation and the conformation reopening, in which the coupling reaction is the rate-limiting step. FIG. 4 suggests that the mismatch base pair would not affect the closing and reopening of a DNA polymerase significantly but affect the enzyme catalyzed transition state (TS). Thus, the modification of the non-base pairing section of naturally occurring nucleosides should be able to tune their kinetic parameters, making them distinguishable by an electronical sensor.

### BRIEF DESCRIPTION OF THE DRAWINGS

FIG. 1: a prior art device composed of a carbon nanotube attached to two electrodes (source and drain) and functionalized with a DNA polymerase for monitoring the enzyme activity in real-time.

FIG. 2: Chemical structure of modified nucleotides α-thio-dATP and 2-thio-dTTP.

FIG. 3: A schematic diagram of a single molecule DNA sequencing device with polymerase on a nanostructure attached to two electrodes, (a) a DNA nanostructure, (b) a peptide nanostructure.

FIG. 4: (a) Free energy profile for single nucleotide incorporation by different DNA polymerase Pol β WT, R258A mutant, KF, and Pol X; (b) Qualitative free energy profile of matched and mismatched dNTP incorporation by Pol β versus I260Q mutant.

FIG. 5: the reaction in incorporating a nucleotide substrate to a DNA chain.

FIG. 6: Chemical structures of naturally occurring nucleoside triphosphates.

FIG. 7: Chemical structures of naturally occurring nucleoside γ-substituted triphosphates.

FIG. 8: Chemical structures of β, γ-X analogies of naturally occurring nucleoside triphosphates.

FIG. 9: Chemical structures of naturally occurring nucleoside α-thio-triphosphates (α-thio-dNTP).

FIG. 10: Chemical structures of naturally occurring nucleoside α-borano-triphosphates (α-borano-dNTP).

FIG. 11: Chemical structures of naturally occurring nucleoside α-borano-α-thio-triphosphates (α-borano-α-thio-dNTPs).

FIG. 12: Chemical structures of naturally occurring nucleoside α-seleno-triphosphates (α-seleno-dNTP).

FIG. 13: Chemical structures of naturally occurring deoxyribonucleoside α-R-phosphonyl-β, γ-diphosphates.

FIG. 14: Chemical structures of naturally occurring nucleoside triphosphates with both oxygen bridges modified (β,γ-X-α-Z-dNTP).

FIG. 15: Chemical structures of naturally occurring nucleoside triphosphates with one of γ-, and α-phosphorus' oxygens replaced by other atoms or organic groups.

FIG. 16: Chemical structures of nucleotide with their sugar ring oxygen replaced by other atoms.

FIG. 17: Chemical structures of representative xeno nucleic acid (XNA) nucleosides.

FIG. 18: Diagram of Watson-Crick base pairs and modification sites in this invention.

FIG. 19: Chemical structures of modified pyrimidine nucleobases.

FIG. 20: Chemical structures of modified purine nucleobases.

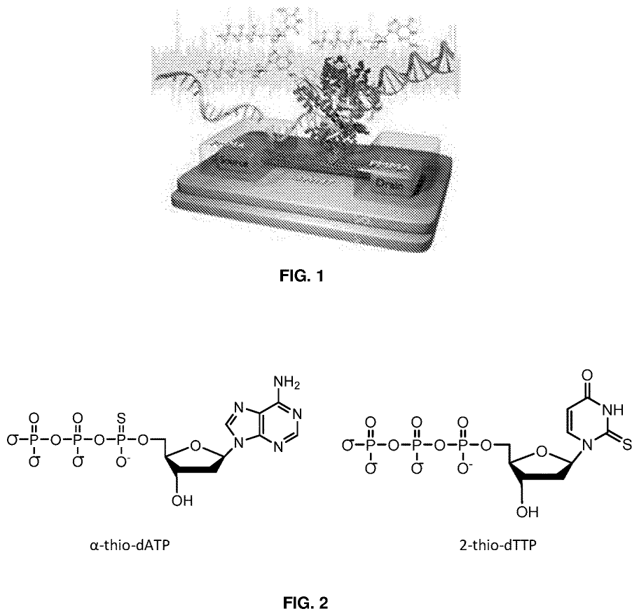

### SUMMARY OF THE INVENTION

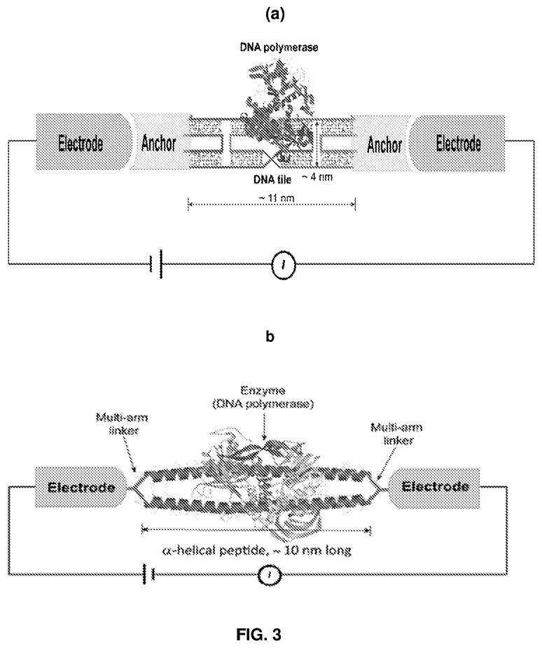

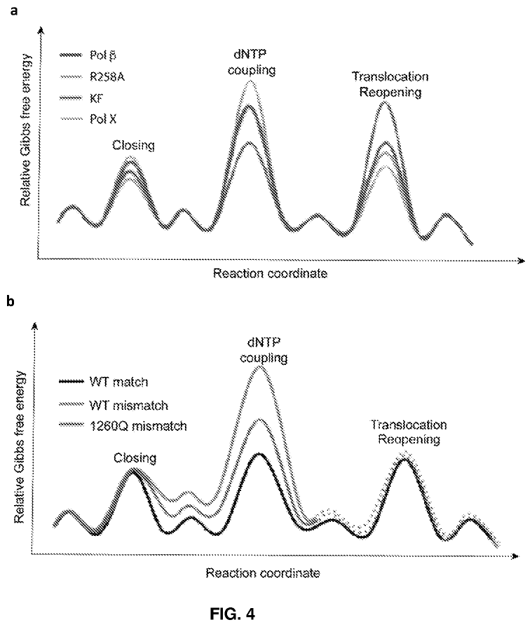

This invention includes a biopolymer nanostructure coupling with a DNA polymerase as an electronic sensor for nucleic acid sequencing (see FIG. 3a, a DNA nanostructure, and FIG. 3b, a peptide nanostructure), as disclosed in the provisional patent applications, U.S. 62/794,096, U.S. 62/812,736, U.S. 62/833,870, and U.S. 62/803,100, which are included herein by their entirety. Both the DNA nanostructure and peptide nanostructure illustrated in FIG. 3 are conductors of electron charges under certain conditions through tunneling and hopping. A DNA polymerase is attached to the nanostructure at the predefined locations, each through a short flexible linker. For sequencing, the DNA polymerase first forms a binary complex with a target-primer duplex, existing in an “open” conformation, which can, in turn, form a ternary complex with a correct nucleoside triphosphate through the Watson-Crick base pairing. In the presence of metal ions, the ternary complex turns the DNA polymerase to a “closed” conformation, facilitating the elongation reaction. When a new phosphodiester bond is formed, the nascent base pair at the end of the duplex is overstretched, which triggers a stacking interaction with the nearest-neighbor base-pair. Such a process shifts DNA and DNA polymerase in opposite directions, hence giving rise to an open conformation for the next round of incorporation.⁵ All of these mechanical movements, including the conformation change and DNA translocation, exert forces on the underneath nanostructure and disturb its base pairing and stacking, resulting in fluctuations in the charge transport as a signature of the nucleotide incorporation.

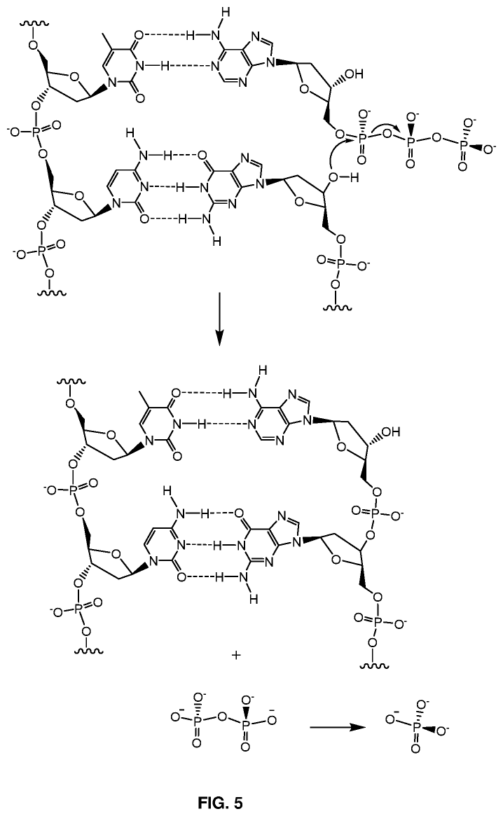

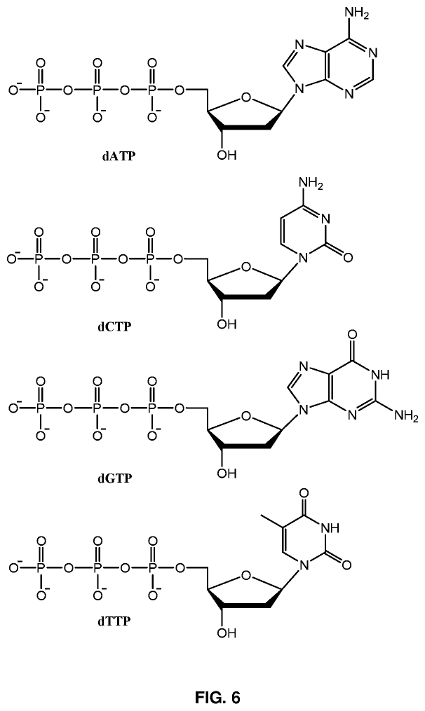

This invention provides methods and chemicals for identifying individual components (or units or bases) that constitute biopolymers, especially DNAs and RNAs. For example, to sequence a target DNA molecule, we use it as a template for DNA synthesis on the said nanostructure sequencing device, with which nucleoside triphosphate substrates are incorporated into a growing DNA strand following the Watson Crick base pairing rule. The DNA sequence is determined by reading the nucleotide incorporation. A recent study has shown that the DNA synthesis is a two Mg²⁺ ion assisted stepwise associative S_{N}2 reaction,⁶ albeit a third divalent metal ion may be present during DNA synthesis.⁷ Moreover, the pyrophosphate (PPi) group released from the S_{N}2 reaction is hydrolyzed to phosphates during the DNA synthesis catalyzed by DNA polymerases. A general mechanism of DNA polymerization is illustrated in FIG. 5. The terminal 3′ oxygen of the growing strand acts as a nucleophile to attack the α-phosphorus atom of the incoming dNTP to forms a P—O covalent bond, accompanied by the release of pyrophosphate that is in turn hydrolyzed to phosphates. Based on the reaction mechanism, this invention provides modified nucleotide substrates, which affect the kinetics of the polymerase enzymatic reactions in ways different from the naturally occurring nucleotides, generating distinguishable electric signals in the nanostructure that can be used to differentiate individual nucleotides in the target DNA template so that the target DNA can be sequenced.

The DNA polymerases used in this invention include those that have been classified by structural homology into the families of A, B, C, D, X, Y, and RT. For example, those in Family A include T7 DNA polymerase and Bacillus stearothermophilus Pol I; those in Family B include T4 DNA polymerase, Phi29 DNA polymerase, and RB69; those in Family C include the E. coli DNA Polymerase III. The RT (reverse transcriptase) family of DNA polymerases include, for example, retrovirus reverse transcriptases and eukaryotic telomerases.

### DETAILED DESCRIPTION

In some embodiments, a polymerase is attached to the nanostructure, fed with a duplex composed of DNA primer and a target to be sequenced, and followed by a mixture of nucleoside triphosphates or dNTPs. In the presence of metal ions, the DNA polymerase incorporates the dNTPs into the DNA primer according to the Watson-Crick pairing rule, and each incorporating step evokes an electric spike that is recorded in the sensor.

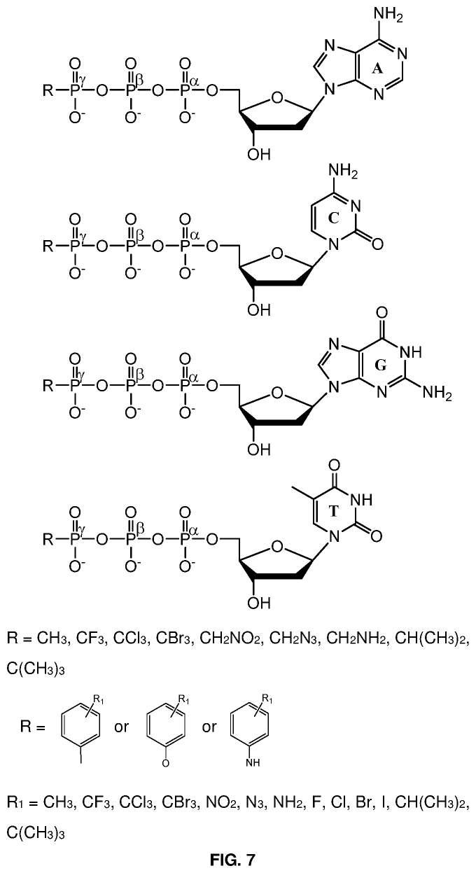

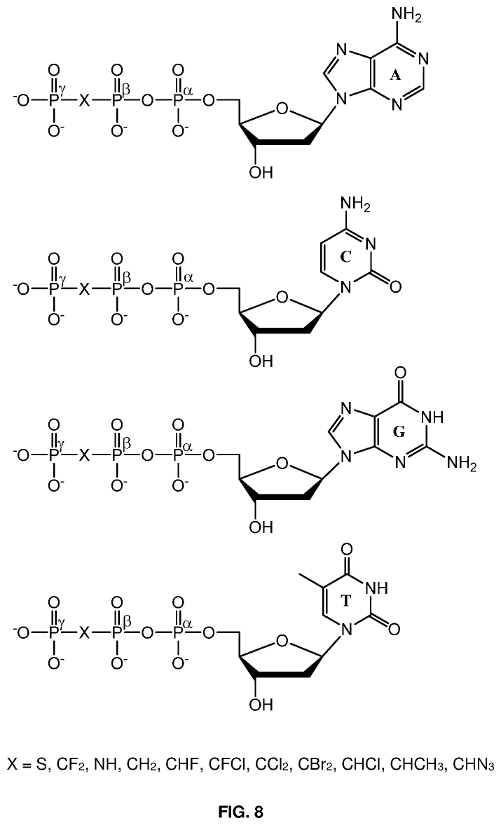

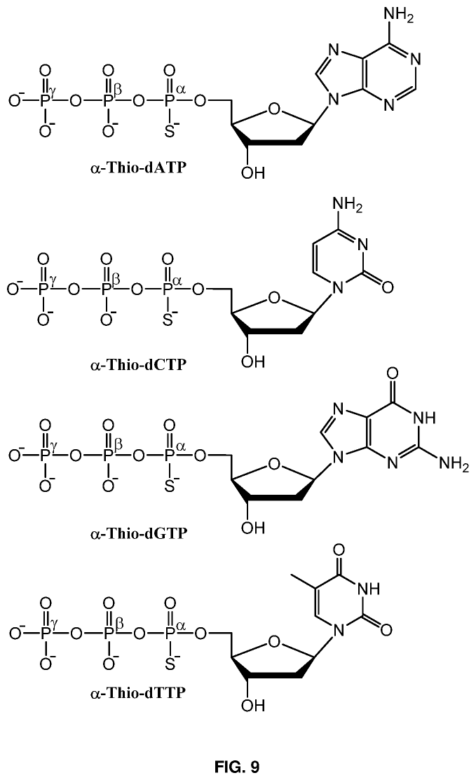

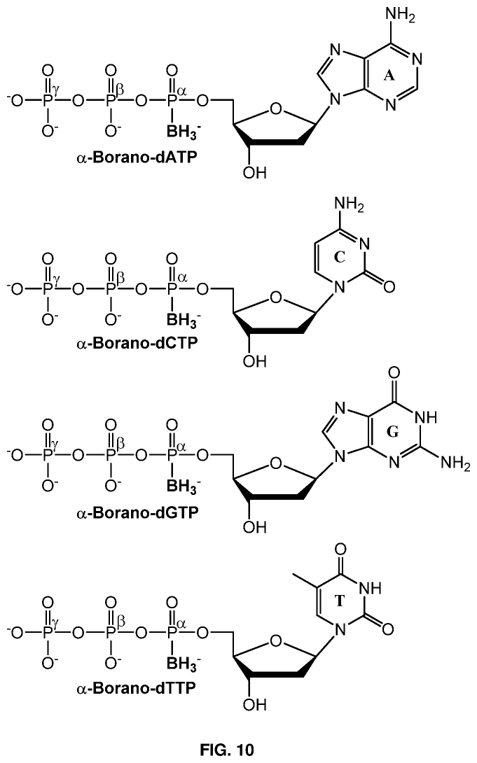

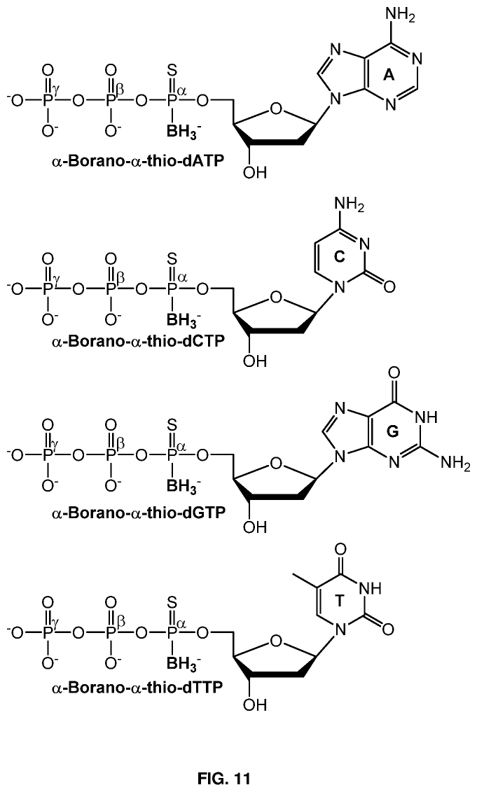

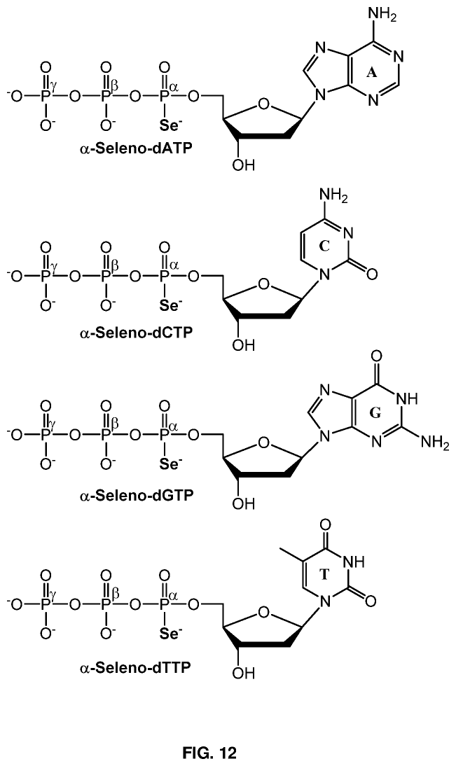

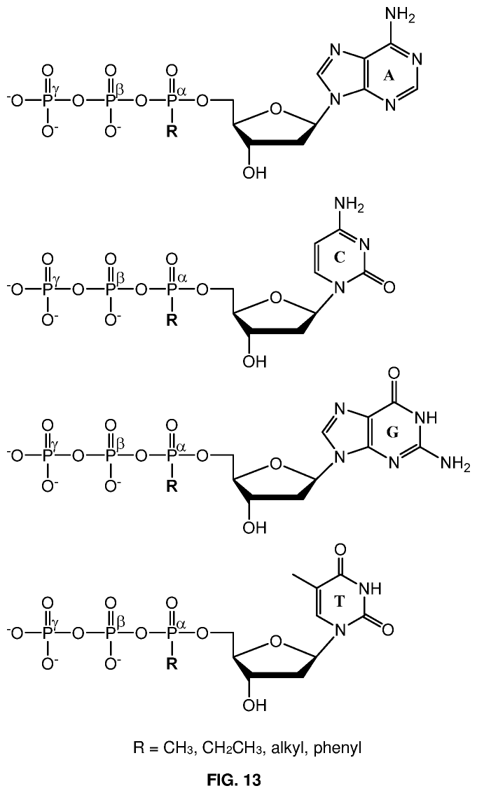

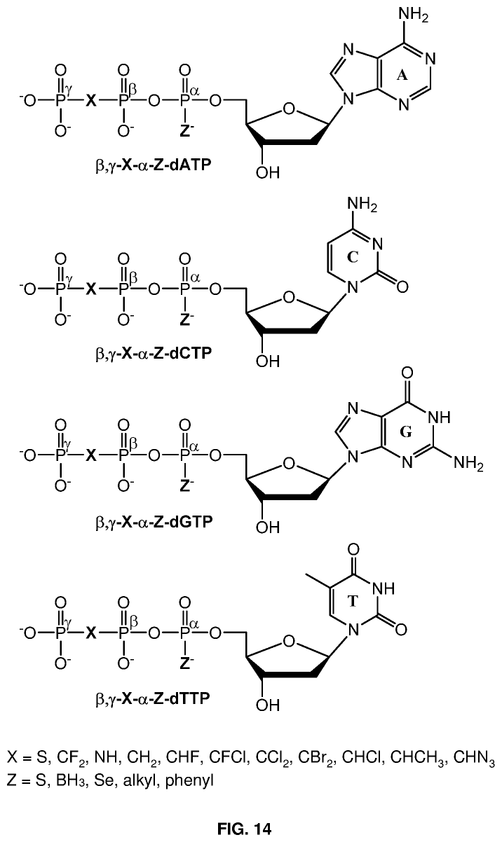

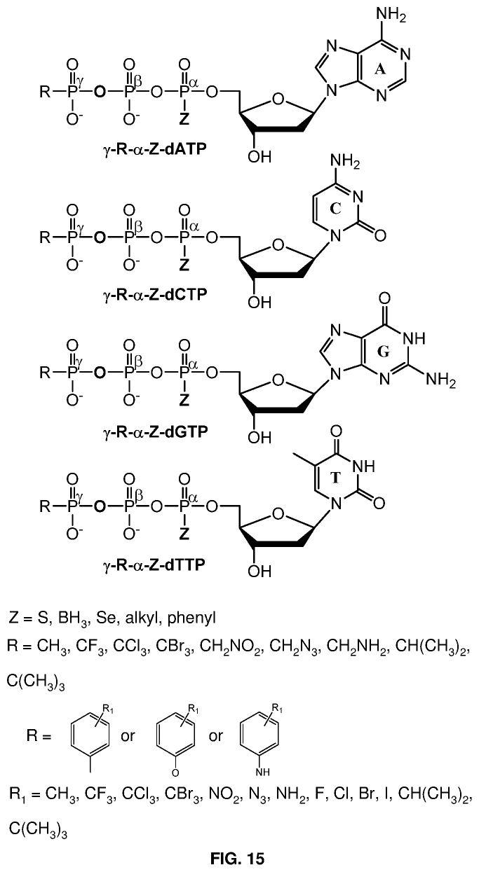

The above said nucleoside triphosphate mixtures include:
0-4 of naturally occurring nucleoside triphosphates (FIG. 6).
0-4 of naturally occurring nucleoside γ-substituted triphosphates (FIG. 7). The substituents are either electron donating or electron withdrawing groups that affect the activities of DNA polymerases,⁸ and also may affect the hydrolysis of pyrophosphate to phosphates, resulting in altered reaction rates and, in turn, the electric signals.
0-4 of β,γ-X analogies of naturally occurring nucleoside triphosphates (FIG. 8). In the analogy, the X moiety substitutes for the β; γ-bridging O of the naturally occurring nucleoside triphosphate, which alters the stereoelectronic properties of the bisphosphonate (BP) leaving group without affecting the base pairing. As a result, these triphosphate analogies modulate the incorporation rates of the DNA polymerase, which is affected by the leaving groups.⁹⁻¹¹ Since the incorporation is a kinetically controlled process, the corresponding electric signals can be modulated accordingly.
0-4 of α-thio-dNTPs (FIG. 9). These modified triphosphates are incorporated into DNA primers by DNA polymerase. The Sₚ-diastereomers of deoxyribonucleoside and ribonucleoside 5′-O-(1-thio-triphosphates) are analogs of the naturally occurring nucleotides and are incorporated readily into nucleic acids by DNA or RNA polymerases.^{12, 13}
0-4 of α-borano-dNTPs (FIG. 10). The α-borano-dNTPs and α-borano-NTPs are good to excellent substrates for DNA and RNA polymerases, allowing for ready enzymatic syntheses of DNA and RNA.^{14, 15}
0-4 of α-borano-α-thio-dNTPs (FIG. 11).¹⁶
0-4 of α-seleno-dNTP (FIG. 12). These modified dNTPs can be incorporated into DNA. However, the DNA polymerization with α-seleno-dNTPs is slower than with the native dNTPs. The α-Seleno-dNTPs suppress the primer self-extension in the lack of a DNA template.¹⁷
0-4 of deoxyribonucleoside α-R-phosphonyl-β, γ-diphosphate (FIG. 13). These substrates produce uncharged nucleic acid backbone¹⁸, which facilitates the further distinction between the electric signals generated from the incorporation of different substrates.
0-4 of β,γ-X-α-Z-dNTP analogs (FIG. 14), which facilitate the further distinction between the electric signals generated from the incorporation of different substrates.
0-4 of γ-R-α-Z-dNTP analogies (FIG. 15), which facilitate the further distinction between the electric signals generated from incorporation of different substrates.

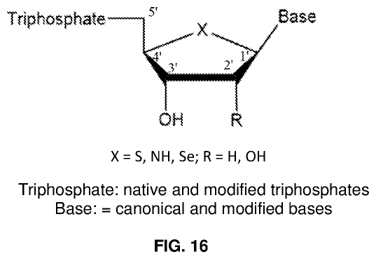

In some embodiments, the said nucleoside triphosphates include modified sugars. FIG. 16 shows one form of the modifications, in which the oxygen in the sugar ring is replaced by another atom. These atoms have different electron negativities, which would affect the pKₐ of the neighbor 3′-OH, and in turn its nucleophilicity. For example, 2′-deoxy-4′-thioribonucleoside 5′-triphosphate (dSNTPs), where X=S, R=H, Base=A, C, G, T, with unmodified triphosphate, can be used as substrates of DNA polymerases.¹⁹ dSNTPs have shown different reaction rates and efficiencies from the corresponding native dNTPs.

In some embodiments, the said nucleoside triphosphates include the ribose sugar (FIG. 16, R=OH). An RNA dependent RNA polymerase (RdRP) is attached to the DNA nanostructure device for RNA sequencing. The enzyme is polio virus RdRP and others.

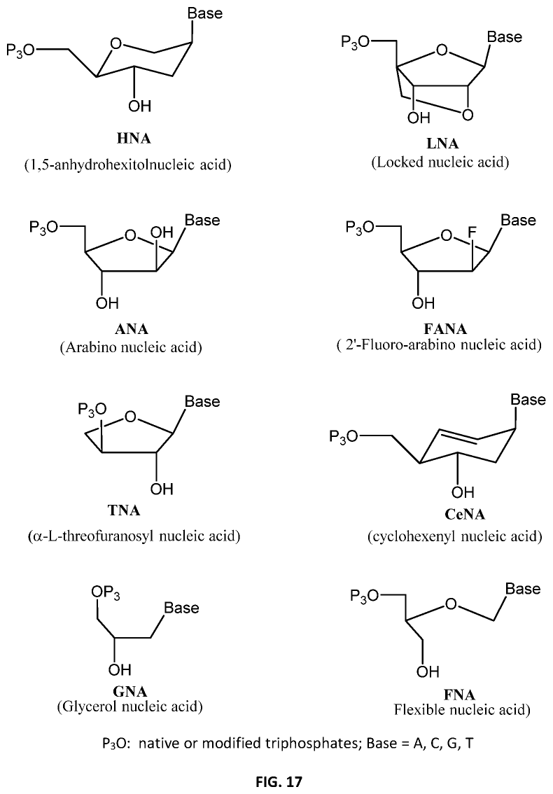

In some embodiments, the said nucleoside triphosphates have the nucleoside units including artificial genetic polymer xeno nucleic acids (XNA), a set of nucleic acid polymers with their backbone structures distinct from those found in nature, which is capable of specifically base pairing with DNA nucleobases (FIG. 17). Some of XNAs have their sugar units flexible or rigid conformations, and others have different configurations and structures from their naturally occurring counterparts. These make their binding to targets in the enzyme differently from those naturally occurring counterparts. Also, some XNAs carry an electron-donating or withdrawing group that make its neighbor OH more or less nucleophilic, compared to the naturally occurring counterpart. For example, TNA can be incorporated into a DNA primer by a laboratory evolved polymerase that derives from a replicative B-family polymerase isolated from the archaeal hyperthermophilic species Thermococcus kodakarensis (Kod).^{20, 21} These XNA substrates are useful to distinguish a specific DNA nucleotide from the rest of them in a DNA target.

In some embodiments, the RNA polymerase attached to the biopolymer nanostructure sensor for RNA sequencing includes, but not limited to, viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA polymerase. The RNA polymerase attached to sensor is fed with a mixture of canonical ribonucleoside triphosphates for reading out RNA sequences.

In some embodiments, the said mixture contains
0-4 of canonical ribonucleoside triphosphates.
0-4 of ribonucleotides with their triphosphates modified (FIG. 16, R=OH)
0-4 of modified ribonucleoside triphosphates with a generalized structure as shown in FIG. 16, where R=OH.
0-4 of XNA triphosphates (FIG. 17).

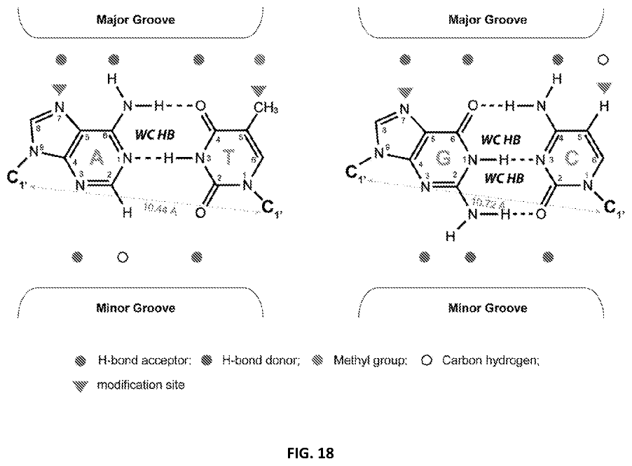

This invention further provides modified bases to further tune both DNA and RNA polymerase for their reactivities. To maintain the fidelity of polymerases, we remain the WC edges unchanged for the modified bases, as shown in FIG. 18. These compounds have a common feature of the preserved Watson-Crick hydrogen bonding edges for inserting a correct incoming nucleotide to interact with the template following the Watson-Crick base pairing rule and hydrogen bonding acceptor sites for a polymerase to interact with the base pair from the minor groove.^{22, 23} Thus, the modifications do not disturb the fidelity of the enzyme.

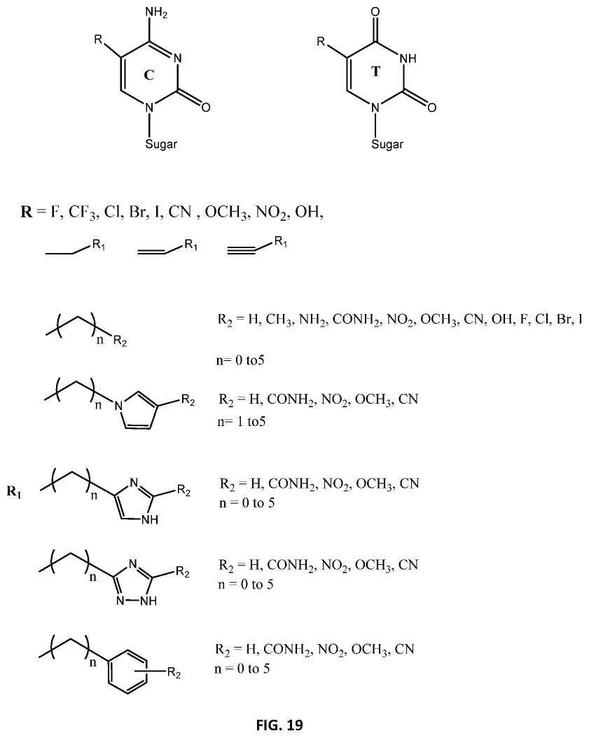

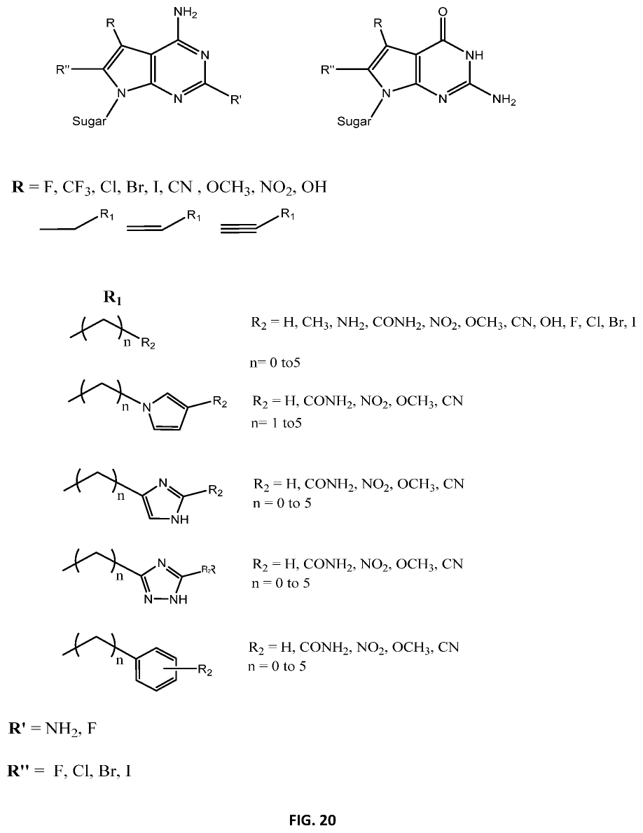

In some embodiments, the said nucleoside triphosphates are composed of the pyrimidine bases with their 5-positions modified with a series of electron-withdrawing groups, electron donor groups, as well as ethyl, ethylene, and acetylene, to which various functional groups are attached (FIG. 19). These modifications allow us to tune the transition state of the enzymatic reaction.

In some embodiments, the said nucleoside triphosphates are composed of the purines bases with their 7-positions modified with a series of electron-withdrawing groups, electron donor groups, as well as ethyl, ethylene, and acetylene, to which various functional groups are attached (FIG. 20). These modifications allow us to tune the transition state of the enzymatic reaction.

In some embodiments, the nucleoside triphosphates are composed of the said modified bases, modified sugars or sugar analogies, and modified triphosphates or triphosphate analogies.

In some embodiments, a plurality of nanostructure sensors are used to read the nucleic acid sequences in parallel. A plurality of nanostructure sensors can be fabricated in an array format with the number of nanostructure sensors from 10⁴ to 10⁹ on a solid surface or in a well, preferably 10³ to 10⁷ or more preferably 10⁴ to 10⁶.

All of the nanostructure sensors in the said array is configured with one type of nucleic acid polymerase or different types of nucleic acid polymerases.

The target sample can be double or single-stranded, linear, or circular DNA.

The target sample can also be double or single-stranded, linear, or circular RNA. The primer for the sequencing can be DNA, RNA, conjugates of DNA and RNA, or DNA containing modified nucleosides.

A polymerase can be attached to a biopolymer nanostructure sensor at a predefined location or locations using the attachments chemistries provided in the previous provisional patent applications (ref. U.S. 62/794,096, U.S. 62/812,736, U.S. 62/833,870, and U.S. 62/803,100). In many embodiments, a DNA nanostructure is functionalized with organic functional groups at the predefined DNA nucleoside or nucleosides. Whereas the DNA polymerase is bioengineered to contain unnatural amino acids that bear the function against those in the DNA nanostructure for the click reaction.

In some embodiments, the biopolymer nanostructure in all the above descriptions is replaced by a solid nanowire made of material selected from the group of platinum (Pt), palladium (Pd), Tungsten (W), gold (Au), copper (Cu), titanium (Ti), Tantalum (Ta), Chromium (Cr), TiN, TiNx, TaN, TaNx, silver (Ag), aluminum (Al), and other metals, preferably Pt, Pd, Au, Ti, and TiN. The nanowire is 3 nm to 10 μm in length, preferably 20 nm to 1 μm; 5 nm to 50 nm in width, preferably 5 nm to 20 nm; and 3 nm to 50 nm in thickness, preferably 4 nm to 10 nm. All the nucleoside triphosphate and ribonucleoside triphosphate designs, modifications, variations, natural or unnatural, their interaction with polymerase, their methods of use, and principles of distinguishing individual nucleotides apply to the polymerase-nanowire coupled DNA/RNA sequencing system. In some other embodiments, the nanowire is a carbon nanotube or a graphene sheet, single layer or multilayer, with dimension similar to the nanowire.

In some embodiments, the nanostructure in all the above descriptions is replaced by a molecular wire, such as those disclosed in patent applications, WO2018208505, US20180305727A1, and WO2018136148A1. All the nucleoside triphosphate and ribonucleoside triphosphate designs, modifications, variations, natural or unnatural, their interaction with polymerase, their methods of use and principles of distinguishing individual nucleotides apply to the polymerase-molecular wire coupled DNA/RNA sequencing system.

In some embodiments, a DNA polymerase is directly attached to the two electrodes, bridging the nanogap between the two electrodes and allowing electrons or electric current to pass through, such as those disclosed in patent applications WO2018208505, US20180305727A1 and WO2018136148A1. For the purpose of DNA/RNA sequencing, the above mentioned nucleoside triphosphate and ribonucleoside triphosphate designs, modifications, variations, natural or unnatural, their interaction with polymerase, their methods of use and principles of distinguishing individual nucleotides apply to the polymerase only DNA/RNA sequencing system.

In some embodiments, all the above-mentioned nanogap bridging configurations, such as the biopolymer nanostructure, the molecular wire, the nanowire and the polymerase directly contacting the nanogap electrodes, can be combined with a gate electrode to form a FET type polymerase sequencing system, such as those disclosed in the provisional patent application U.S. 62/833,870. Although the mechanism of polymerase conformational change affecting the electrical signal passing through the nanogap is somehow different, the nucleoside triphosphate and ribonucleoside triphosphate designs, modifications, variations, natural or unnatural, and their interaction with polymerase, their methods of use and principles of distinguishing individual nucleotides also apply to the FET type polymerase DNA/RNA sequencing system.

General Remarks:

Patents or patent applications are incorporated into where they are mentioned in the text. The cited journal publications are listed in Cited Literature.

Unless defined otherwise, all technical and scientific terms used herein take on the meaning commonly understood by one of the ordinary skills in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of applicant's general inventive concept.

### CITED REFERENCES

1. Olsen, T. J.; Choi, Y.; Sims, P. C.; Gul, 0. T.; Corso, B. L.; Dong, C.; Brown, W. A.; Collins, P. G.; Weiss, G. A., Electronic Measurements of Single-Molecule Processing by DNA Polymerase I (Klenow Fragment). J. Am. Chem. Soc. 2013, 135, 7855-7860.
2. Pugliese, K. M.; Gul, 0. T.; Choi, Y.; Olsen, T. J.; Sims, P. C.; Collins, P. G.; Weiss, G. A., Processive Incorporation of Deoxynucleoside Triphosphate Analogs by Single-Molecule DNA Polymerase I (Klenow Fragment) Nanocircuits. J Am Chem Soc 2015, 137, 9587-9594.
3. Roettger, M. P.; Bakhtina, M.; Tsai, M.-D., Mismatched and Matched dNTP Incorporation by DNA Polymerase Proceed via Analogous Kinetic Pathways. Biochemistry 2008, 47, 9718-9727.
4. Bakhtina, M.; Roettger, M. P.; Kumar, S.; Tsai, M.-D., A Unified Kinetic Mechanism Applicable to Multiple DNA Polymerases. Biochemistry 2007, 46, 5463-5472.
5. Arias-Gonzalez, J. R., A DNA-centered explanation of the DNA polymerase translocation mechanism. Scientific reports 2017, 7, 7566.
6. Kottur, J.; Nair, D. T., Pyrophosphate hydrolysis is an intrinsic and critical step of the DNA synthesis reaction. Nucleic Acids Research 2018, 46, 5875-5885.
7. Raper, A. T.; Reed, A. J.; Suo, Z., Kinetic Mechanism of DNA Polymerases: Contributions of Conformational Dynamics and a Third Divalent Metal Ion. Chem Rev 2018, 118, 6000-6025.
8. Arzumanov, A. A.; Semizarov, D. G.; Victorova, L. S.; Dyatkina, N. B.; Krayevsky, A. A., g-Phosphate-substituted 2′-Deoxynucleoside 5′-Triphosphates as Substrates for DNA Polymerases. THE JOURNAL OF BIOLOGICAL CHEMISTRY 1996, 271, 24389-24394.
9. Sucato, C. A.; Upton, T. G.; Kashemirov, B. A.; Osuna, J.; Oertell, K.; Beard, W. A.; Wilson, S. H.; Florian, J.; Warshel, A.; McKenna, C. E.; Goodman, M. F., DNA Polymerase β Fidelity: Halomethylene-Modified Leaving Groups in Pre-Steady-State Kinetic Analysis Reveal Differences at the Chemical Transition State. Biochemistry 2008, 47, 870-879.
10. Oertell, K.; Chamberlain, B. T.; Wu, Y.; Ferri, E.; Kashemirov, B. A.; Beard, W. A.; Wilson, S. H.; McKenna, C. E.; Goodman, M. F., Transition State in DNA Polymerase β Catalysis: Rate-Limiting Chemistry Altered by Base-Pair Configuration. Biochemistry 2014, 53, 1842-1848.
11. Oertell, K.; Florián, J.; Haratipour, P.; Crans, D. C.; Kashemirov, B. A.; Wilson, S. H.; McKenna, C. E.; Goodman, M. F., A Transition-State Perspective on Y-Family DNA Polymerase η Fidelity in Comparison with X-Family DNA Polymerases λ and β. Biochemistry 2019, 58, 1764-1773.
12. Eckstein, F., NUCLEOSIDE PHOSPHOROTHIOATES. Ann. Rev. Biochem. 1985, 54, 367-402.
13. GISH, G.; ECKSTEIN, F., DNA and RNA Sequence Determination Based on Phosphorothioate Chemistry. Science 240, 1520-1521.
14. Russell, C.; Roy, S.; Ganguly, S.; Qian, X.; Caruthers, M. H.; Nilsson, M., Formation of Silver Nanostructures by Rolling Circle Amplification Using Boranephosphonate-Modified Nucleotides. Analytical Chemistry 2015, 87, 6660-6666.
15. SHAW, B. R.; DOBRIKOV, M.; WANG, X.; WAN, J.; HE, K.; LIN, J.-L.; LI, P.; RAIT, V.; SERGUEEVA, Z. A.; SERGUEEV, D., Reading, Writing, and Modulating Genetic Information with Boranophosphate Mimics of Nucleotides, DNA, and RNA. Annals of the New York Academy of Sciences 2003, 1002, 12-29.
16. Lin, J.; Ramsay Shaw, B., Synthesis of a novel triphosphate analogue: nucleoside α-borano, α-thiotriphosphate. Chemical Communications 2000, (21), 2115-2116.
17. Hu, B.; Wang, Y.; Sun, S.; Yan, W.; Zhang, C.; Luo, D.; Deng, H.; Hu, L. R.; Huang, Z., Synthesis of Selenium-Triphosphates (dNTPαSe) for More Specific DNA Polymerization. Angewandte Chemie International Edition 2019, 58, 7835-7839.
18. Arangundy-Franklin, S.; Taylor, A. I.; Porebski, B. T.; Genna, V.; Peak-Chew, S.; Vaisman, A.; Woodgate, R.; Orozco, M.; Holliger, P., A synthetic genetic polymer with an uncharged backbone chemistry based on alkyl phosphonate nucleic acids. Nature Chemistry 2019, 11, 533-542.
19. Kojima, T.; Furukawa, K.; Maruyama, H.; Inoue, N.; Tarashima, N.; Matsuda, A.; Minakawa, N., PCR amplification of 4′-thioDNA using 2′-deoxy-4′-thionucleoside 5′-triphosphates. ACS Synth Biol 2013, 2, 529-36.
20. Dunn, M. R.; Otto, C.; Fenton, K. E.; Chaput, J. C., Improving Polymerase Activity with Unnatural Substrates by Sampling Mutations in Homologous Protein Architectures. ACS Chemical Biology 2016, 11, 1210-1219.
21. Chim, N.; Shi, C.; Sau, S. P.; Nikoomanzar, A.; Chaput, J. C., Structural basis for TNA synthesis by an engineered TNA polymerase. Nature Communications 2017, 8, 1810.
22. Freudenthal, B. D.; Beard, W. A.; Wilson, S. H., Structures of dNTP intermediate states during DNA polymerase active site assembly. Structure 2012, 20, 1829-1837.
23. Xia, S.; Konigsberg, W. H., RB69 DNA Polymerase Structure, Kinetics, and Fidelity. Biochemistry 2014, 53, 2752-2767.

What is claimed:

1. A system for identification, characterization, or sequencing of a biopolymer comprising,
(a) a non-conductive substrate;
(b) a nanogap formed by a first electrode and a second electrode placed next to each other on the non-conductive substrate;
(c) a nanostructure configured to have a dimension comparable to the nanogap and to bridge the nanogap by attaching one end to the first electrode and another end to the second electrode through a chemical bond;
(d) a DNA or an RNA polymerase attached to the nanostructure and configured to perform a biopolymer synthesis reaction;
(e) a reaction mixture that facilitates the biopolymer synthesis reaction;
(f) a bias voltage that is applied between the first electrode and the second electrode;
(g) a device that records a current fluctuation through the nanostructure resulting from a distortion within the nanostructure caused by a conformation change initiated by the polymerase attached to the nanostructure; and
(h) a software for data analysis that identifies the biopolymer or a subunit of the biopolymer;
wherein the biopolymer is either a DNA molecule, a RNA molecule, or a oligonucleotide, or a combination thereof, and either double or single stranded, linear or circular, natural, modified or synthesized, and a combination thereof.

2. The system of claim 1, wherein the non-conductive substrate comprises the following: silicon, silicon oxide, silicon nitride, glass, hafnium dioxide, other metal oxides, any non-conductive polymer film, silicon with silicon oxide or silicon nitride or other non-conductive coatings, glass with silicon nitride coating, other non-conductive organic materials, and/or any non-conductive inorganic materials.

3. The system of claim 1, wherein the nanostructure is one of the following or a combination thereof:
(a) a DNA nanostructure, made of deoxyribonucleic acid, either natural, modified or synthesized;
(b) an RNA nanostructure, made of ribonucleic acid, either natural, modified or synthesized;
(c) a peptide nanostructure, made of amino acid, either natural, modified or synthesized; and
(d) a molecular wire made of any conductive biopolymer or biopolymers, either natural, modified or synthesized.

4. The system of claim 1, wherein the nanostructure comprises a solid nanowire made of a metal material selected from the group consisting of platinum (Pt), palladium (Pd), Tungsten (W), gold (Au), copper (Cu), titanium (Ti), Tantalum (Ta), Chromium (Cr), TiN, TiNx, TaN, TaNx, silver (Ag), aluminum (Al), and other metals, and a combination thereof.

5. The system of claim 1, wherein the nanostructure comprises a carbon nanotube or a graphene sheet, either a single layer or multilayer or a combination thereof.

6. The system of claim 1, wherein the nanostructure is the polymerase wherein the polymerase is directly attached to the two electrodes, bridging the nanogap, and allowing the electronic current to pass through.

7. The system of claim 1, wherein the DNA polymerase is selected from the group consisting of DNA polymerase families A, B, C, D, X, Y, and RT, comprising T7 DNA polymerase, Phi29 DNA polymerase, Taq polymerase, DNA polymerase Y, DNA Polymerase Pol I, Pol II, Pol III, Pol IV, and Pol V, Pol α (alpha), Pol β (beta), Pol σ (sigma), Pol λ (lambda), Pol δ (delta), Pol ε (epsilon), Pol μ(mu), Pol I (iota), Pol κ (kappa), pol η (eta), and terminal deoxynucleotidyl transferase, telomerase, either native, mutated, expressed, or synthesized, and a combination thereof.

8. The system of claim 1, wherein the RNA polymerase is selected from the group consisting of viral RNA polymerases, comprising T7 RNA polymerase; and Eukaryotic RNA polymerases, comprising RNA polymerase I, RNA polymerase II, RNA polymerase III, RNA polymerase IV, and RNA polymerase V; and Archaea RNA polymerase, either native, mutated, expressed, or synthesized, and a combination thereof.

9. The system of claim 1, further comprising a third electrode, configured to function as a gate electrode, wherein together with the first and the second electrodes, they form a FET type nanogap device.

10. The system of claim 1, wherein the reaction mixture comprises at least one of the following nucleoside triphosphate mixtures or a combination thereof:
(a) at least a naturally occurring nucleoside triphosphates;
(b) at least a naturally occurring nucleoside γ-substituted triphosphates, comprising either electron-donating or electron-withdrawing groups;
(c) at least a β,γ-X analogs of naturally occurring nucleoside triphosphates with the X moiety substituting for the β,γ-bridging O of the naturally occurring nucleoside triphosphate;
(d) at least a α-thio-dNTPs or α-thio-NTPs;
(e) at least a α-borano-dNTPs or α-borano-NTPs;
(f) at least a α-borano-α-thio-dNTPs or α-borano-α-thio-NTPs;
(g) at least a α-seleno-dNTPs or α-seleno-NTPs;
(h) at least a deoxyribonucleoside α-R-phosphonyl-β, γ-diphosphate;
(i) at least a β,γ-X-α-Z-dNTP analogies or β,γ-X-α-Z-NTP analogies; and
(j) at least a γ-R-α-Z-dNTP analogies or γ-R-α-Z-NTP analogies,
wherein the dNTP is configured for DNA synthesis and the NTP is configured for RNA synthesis.

11. The system of claim 1, wherein the reaction mixture comprises at least one of the following or a combination thereof:
(a) a dNTP or a NTP that comprises a modified sugar, wherein the oxygen in the sugar ring is replaced by an atom that has a different electron negativity;
(b) a dNTP or a NTP that comprises a nucleoside unit comprising an artificial genetic polymer xeno nucleic acid (XNA);
(c) a dNTP or a NTP that comprises a pyrimidine base with the 5-position modified with a molecule selected from the group consisting of an electron-withdrawing group, an electron-donor groups, an ethyl group, an ethylene group, an acetylene group, and a combination thereof, to which a functional group is attached; and
(d) a dNTP or a NTP that comprises a purine base with the 7-position modified with a molecule selected from the group consisting of an electron-withdrawing group, an electron-donor group, an ethyl group, an ethylene, and an acetylene group, and a combination thereof, to which a functional group is attached; and
wherein the dNTP is configured for DNA synthesis and the NTP is configured for RNA synthesis.

12. The system of claim 1 comprises a plurality of nanogap sensors, wherein each nanogap sensor comprises a pair of electrodes, a nanostructure, a polymerase, a reaction mixture, and any feature associated with a single nanogap.

13. The system of claim 12, wherein the plurality of nanogap sensors comprise an array of about 10 to about 1 billion nanogaps, preferably between about 10,000 to about 1 million nanogaps.

14. A method for identification, characterization, or sequencing of a biopolymer comprising,
(a) providing a non-conductive substrate;
(b) providing a first electrode and a second electrode, and placing them next to each other to form a nanogap on the non-conductive substrate;
(c) providing a nanostructure configured to have a dimension comparable to the nanogap and to bridge the nanogap by attaching one end to the first electrode and another end to the second electrode through a chemical bond;
(d) providing a DNA or RNA polymerase attached to the nanostructure and configured to perform a biopolymer synthesis reaction;
(e) providing a reaction mixture that facilitates the biopolymer synthesis reaction;
(f) applying a bias voltage between the first electrode and the second electrode;
(g) recording the current fluctuation through the nanostructure resulting from a distortion within the nanostructure caused by a conformation change initiated by the polymerase attached to the nanostructure; and
(h) providing a software for data analysis that identifies the biopolymer or a subunit of the biopolymer; and
wherein the biopolymer is either a DNA molecule, a RNA molecule, or a oligonucleotide, or a combination thereof, and either double or single stranded, linear or circular, natural, modified or synthesized, and a combination thereof.

15. The method of claim 14, wherein the non-conductive substrate comprises the following: silicon, silicon oxide, silicon nitride, glass, hafnium dioxide, other metal oxides, any non-conductive polymer film, silicon with silicon oxide or silicon nitride or other non-conductive coatings, glass with silicon nitride coating, other non-conductive organic materials, and/or any non-conductive inorganic materials.

16. The method of claim 14, wherein the nanostructure is one of the following or a combination thereof:
(a) a DNA nanostructure, made of deoxyribonucleic acid, either natural, modified or synthesized;
(b) an RNA nanostructure, made of ribonucleic acid, either natural, modified or synthesized;
(c) a peptide nanostructure, made of amino acid, either natural, modified or synthesized; and
(d) a molecular wire, made of any conductive biopolymer or biopolymers, either natural, modified or synthesized.

17. The method of claim 14, wherein the nanostructure comprises a solid nanowire made of a metal material selected from the group consisting of platinum (Pt), palladium (Pd), Tungsten (W), gold (Au), copper (Cu), titanium (Ti), Tantalum (Ta), Chromium (Cr), TiN, TiNx, TaN, TaNx, silver (Ag), aluminum (Al), and other metals, and a combination thereof.

18. The method of claim 14, wherein the nanostructure comprises a carbon nanotube or a graphene sheet, either a single layer or multilayer or a combination thereof.

19. The method of claim 14, wherein the nanostructure is the polymerase wherein the polymerase is directly attached to the two electrodes, bridging the nanogap, and allowing the electronic current to pass through.

20. The method of claim 14, wherein the DNA polymerase is selected from the group consisting of DNA polymerase families A, B, C, D, X, Y, and RT, comprising T7 DNA polymerase, Phi29 DNA polymerase, Tag polymerase, DNA polymerase Y, DNA Polymerase Pol I, Pol II, Pol III, Pol IV, and Pol V, Pol α (alpha), Pol β (beta), Pol σ (sigma), Pol λ (lambda), Pol δ (delta), Pol ε (epsilon), Pol μ(mu), Pol I (iota), Pol κ (kappa), pol η (eta), and terminal deoxynucleotidyl transferase, telomerase, either native, mutated, expressed, or synthesized, and a combination thereof.

21. The method of claim 14, wherein the RNA polymerase is selected from the group consisting of viral RNA polymerases comprising T7 RNA polymerase; and Eukaryotic RNA polymerases comprising RNA polymerase I, RNA polymerase II, RNA polymerase III, RNA polymerase IV, and RNA polymerase V; and Archaea RNA polymerase, either native, mutated, expressed, or synthesized, and a combination thereof.

22. The method of claim 14, further comprising providing a third electrode, configured to function as a gate electrode, wherein together with the first and the second electrodes, they form a FET type nanogap device.

23. The method of claim 14, wherein the reaction mixture comprises at least one of the following nucleoside triphosphate mixtures or a combination thereof:
(a) at least a naturally occurring nucleoside triphosphates;
(b) at least a naturally occurring nucleoside γ-substituted triphosphates, containing either electron-donating or electron-withdrawing groups;
(c) at least a β,γ-X analogs of naturally occurring nucleoside triphosphates with the X moiety substituting for the β,γ-bridging O of the naturally occurring nucleoside triphosphate;
(d) at least a α-thio-dNTPs or α-thio-NTPs;
(e) at least a α-borano-dNTPs or α-borano-NTPs;
(f) at least a α-borano-α-thio-dNTPs or α-borano-α-thio-NTPs;
(g) at least a α-seleno-dNTPs or α-seleno-NTPs;
(h) at least a deoxyribonucleoside α-R-phosphonyl-β, γ-diphosphate;
(i) at least a β,γ-X-α-Z-dNTP analogies or β,γ-X-α-Z-NTP analogies; and
(j) at least a γ-R-α-Z-dNTP analogies or γ-R-α-Z-NTP analogies; and
wherein the dNTP is configured for DNA synthesis and the NTP is configured for RNA synthesis.

24. The method of claim 14, wherein the reaction mixture contains at least one of the following or a combination thereof:
(a) a dNTP or a NTP that comprises a modified sugar wherein the oxygen in the sugar ring is replaced by an atom that has different electron negativity;
(b) a dNTP or a NTP that comprises a nucleoside unit comprising an artificial genetic polymer xeno nucleic acid (XNA);
(c) a dNTP or a NTP that comprises a pyrimidine base with the 5-position modified with a molecule selected from the group consisting of an electron-withdrawing group, an electron-donor groups, an ethyl group, an ethylene group, and an acetylene group, and a combination thereof, to which a functional group is attached; and
(d) a dNTP or a NTP that comprises a purine base with the 7-position modified with a molecule selected from the group consisting of an electron-withdrawing group, an electron-donor group, an ethyl group, an ethylene group, and an acetylene group, to which a functional group is attached,
wherein the dNTP is configured for DNA synthesis and the NTP is configured for RNA synthesis.
